(19) United States
(12) Patent Application Publication
(10) Pub. No.: US 2005/0282210 A1
(43) Pub. Date: Dec. 22, 2005

(54) Vaccination of skunks and/or mongooses against rabies

(75) Inventor: Joanne L. Maki, Colbert, GA (US)

Correspondence Address: Judy Jarecki-Black, Ph.D,J.D, 3239 Satellite Blvd., Duluth, GA, 30096, US

(73) Assignee: Merial Limited

(21) Appl. No.: 11/156,829

(22) Filed: Jun. 20, 2005

Related U.S. Application Data
(60) Provisional application No. 60/581,698, filed on Jun. 21, 2004.
(60) Provisional application No. 60/627,878, filed on Nov. 15, 2004.

(51) Int. Cl. F23J 13/08; C12Q 1/68; F23J 13/06; A61K 31/70; A01N 43/04; A61K 39/12

(52) U.S. Cl. 435/6; 454/5

(57) ABSTRACT

The present invention relates to recombinant anti-rabies vaccines and the oral administration of such vaccines to skunks and/or mongooses. Advantageously, the anti-rabies vaccine may comprise a recombinant vaccinia virus containing a rabies glycoprotein gene. The invention encompasses methods of vaccinating skunks and/or mongooses by administration of an anti-rabies vaccines which may comprise a recombinant vaccinia virus containing a rabies glycoprotein gene.

### INCORPORATION BY REFERENCE

This application claims benefit of U.S. Provisional patent application Ser. No. 60/581,698 filed Jun. 21, 2004 and U.S. Provisional patent application Ser. No. 60/627,878 filed Nov. 15, 2004.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates to recombinant anti-rabies vaccines and the administration of such vaccines to skunks and/or mongooses.

### BACKGROUND OF THE INVENTION

Rabies is a disease that can occur in all warm-blooded species and is caused by rabies virus. Infection with rabies virus followed by the outbreak of the clinical features in nearly all instances results in death of the infected species. In Europe, the USA and Canada wild life rabies still exists and is an important factor in the cause of most human rabies cases that occur. On the other hand, urban rabies constitutes the major cause of human rabies in developing countries.

Rabies virus is a non-segmented negative-stranded RNA virus of the Rhabdoviridae family. Rabies virus virions are composed of two major structural components: a nucleocapsid or ribonucleoprotein (RNP), and an envelope in the form of a bilayer membrane surrounding the RNP core. The infectious component of all Rhabdoviruses is the RNP core which consists of the RNA genome encapsidated by the nucleocapsid (N) protein in combination with two minor proteins, i.e. RNA-dependent RNA-polymerase (L) and phosphoprotein (P). The membrane surrounding the RNP core consists of two proteins: a trans-membrane glycoprotein (G) and a matrix (M) protein located at the inner site of the membrane.

The G protein, also referred to as spike protein, is responsible for cell attachment and membrane fusion in rabies virus and additionally is the main target for the host immune system. The amino acid region at position 330 to 340 (referred to as antigenic site III) of the G protein has been identified to be responsible for the virulence of the virus, in particular the Arg residue at position 333. All rabies virus strains have this virulence determining antigenic site III in common.

Raboral V-RG was developed as an alternative rabies vaccine by Merial, Ltd. As an alternative rabies vaccine that proved to have the unique and novel attribute of being effective by the oral route (reviewed by Mackowiak et al., Adv Vet Med. 1999;41:571-83). The vaccine consists of a modified live vaccinia virus containing the rabies surface glycoprotein gene inserted inits genome. The first experimental use of the recombinant vaccine in wildlife was initiated in Europe. The vaccine was contained within a plastic sachet surrounded by an edible fishmeal bait and deployed into areas known to contain rabies-infected red fox populations. These campaigns resulted in a dramatic reduction in rabies cases in red foxes and the use of Raboral V-RG was considered a success. Raboral V-RG was also found to be effective in causing a reduction in rabies in raccoons, coyotes and red foxes (reviewed by Mackowiak et al., Adv Vet Med. 1999;41:571-83).

Despite the success of oral vaccination of wildlife, such as foxes and raccoons, oral vaccination of skunks has been less successful. Rupprecht et al. reported that oral administration of SAD-B19 and ERA/BHK-21 vacccines induced neither seroconversion nor significant protection against rabies challenge (see, e.g., Rupprecht et al., J Wildl Dis. January 1990;26(1):99-102). Rupprecht et al. concluded that their experimental results definitively confirmed previous suggestions of the general inadequacy of several conventional attenuated rabies vaccines given orally to skunks, even at dosages 1,000-fold in excess of those found minimally protective for foxes (see, e.g., Rupprecht et al., J Wildl Dis. January 1990;26(1):99-102).

In a similar study, Vos et al. studied direct oral administration of the modified live rabies virus vaccine, SAD B19, to striped skunks (Mephitis mephitis) (see, e.g., Vos et al., J Wildl Dis. April 2002;38(2):428-31). In this study, three of seven vaccinated skunks seroconverted and none of the control animals had detectable levels of rabies virus neutralizing antibodies (see, e.g., Vos et al., J Wildl Dis. April 2002;38(2):428-31).

In another study, Hanlon et al. evaluates a highly attenuated rabies virus vaccine, SAG-2, by an oral route in skunks and raccoons (see, e.g., Hanlon et al., J Wildl Dis. April 2002;38(2):420-7). Two of five skunks and three of five raccoons developed virus neutralizing antibodies (VNA) by day 14 following oral administration of SAG-2 vaccine. All animals remained healthy. Upon challenge, naive controls succumbed to rabies. Among vaccinated animals, four of five skunks and all five raccoons had VNA on day 7 post-challenge and all survived. Hanlon et al. suggests that SAG-2 is a promising candidate vaccine that may satisfy both safety and efficacy concerns for oral rabies immunization of major North American rabies reservoirs (see, e.g., Hanlon et al., J Wildl Dis. April 2002;38(2):420). However, SAG-2, an attenuated rabies virus mutant has the potential to revert to the pathogenic parental strain (see, e.g., European patent application 583998).

Accordingly, there is a need in the art for an efficacious reliable oral rabines vaccine for administration to skunks and/or mongooses, especially since skunks and mongooses remain a major major rabies reservoir species in North America.

Citation or identification of any document in this application is not an admission that such document is available as prior art to the present invention.

### SUMMARY OF THE INVENTION

The invention is based, in part, on the unexpected and surprising result that Raboral V-RG is effective for the oral vaccination of skunks.

The invention relates to a method of eliciting an immune response in a skunk or mongoose which may comprise administering a composition which may comprise a viral vector which may comprise a rabies surface glycoprotein gene inserted into the viral vector genome in an amount effective for eliciting an immune response in the skunk or mongoose.

In one embodiment, the vector may comprise a modified live vaccinia virus. In another embodiment, the rabies surface glycoprotein gene may be rabies glycoprotein G, which is derived from an ERA strain in one embodiment.

In another embodiment, the vaccinia virus or the vaccinia virus vector may be a Copenhagen strain or a derivative thereof. In another embodiment, the vaccinia virus or the vaccinia virus vector may have a tk⁻ phenotype. In an advantageous embodiment, the vaccinia virus or the vaccinia virus vector may be a Copenhagen strain (or a derivative thereof) and has a tk⁻ phenotype.

In an advantageous embodiment, the modified live vaccinia virus may be Raboral V-RG.

In a particularly advantageous embodiment, administration of the above-described compositions may be oral. Advantageously, the oral administration may be by a bait drop. In one embodiment, the bait drop may comprise a hollow plastic packet. In another embodiment, the composition may be inserted in the hollow polymer cube.

In yet another advantageous embodiment, administration of the above-described compositions may be nasal or through contact with the nasal mucosa.

The invention also encompasses a method for inducing an immunological or protective response in a skunk or mongoose which may comprise administering a composition which may comprise a viral vector which may comprise a rabies surface glycoprotein gene inserted into the viral vector genome in an amount effective for eliciting an immune response in the skunk or mongoose.

In one embodiment, the vector may comprise a modified live vaccinia virus. In another embodiment, the rabies surface glycoprotein gene may be rabies glycoprotein G, which is derived from an ERA strain in one embodiment.

In another embodiment, the vaccinia virus or the vaccinia virus vector may be a Copenhagen strain or a derivative thereof. In another embodiment, the vaccinia virus or the vaccinia virus vector may have a tk⁻ phenotype. In an advantageous embodiment, the vaccinia virus or the vaccinia virus vector may be a Copenhagen strain (or a derivative thereof) and has a tk⁻ phenotype.

In an advantageous embodiment, the modified live vaccinia virus may be Raboral V-RG.

In a particularly advantageous embodiment, administration of the above-described compositions may be oral. Advantageously, the oral administration may be by a bait drop. In one embodiment, the bait drop may comprise a hollow plastic packet. In another embodiment, the composition may be inserted in the hollow polymer cube.

In yet another advantageous embodiment, administration of the above-described compositions may be nasal or through contact with the nasal mucosa.

The invention also provides for a kit for performing any of the above described methods comprising the any of the above described compositions and optionally, instructions for performing the method.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 shows a fishmeal polymer-based bait containing vaccine and a coated sachet containing vaccine.

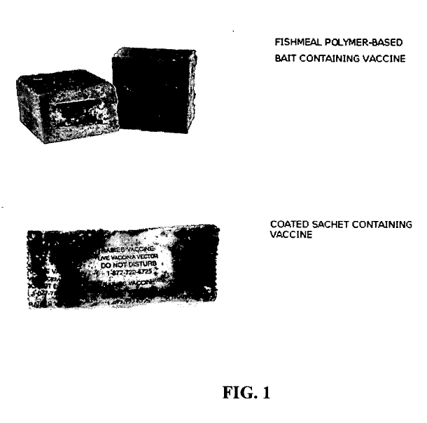

### DETAILED DESCRIPTION

The invention is based, in part, on the unexpected and surprising result that Raboral V-RG is efficacious for the oral vaccination of skunks. Therefore, the invention encompasses, in part, the oral administration of a vaccinia virus vector containing a rabies glycoprotein gene to skunks.

The methods and compositions disclosed herein advantageously relate to the vaccination of skunks and/or mongooses against rabies, however, the methods and compositions may also apply to animals of the Mustilidae, Mephitidae or Viverridae families such as, but not limited to, civits, ferrets, hyenas, lemurs, meerkats, minks and weasels.

In an embodiment of the invention, a rabies glycoprotein gene is encoded into an expression vector. In an advantageous embodiment, the rabies glycoprotein gene is glycoprotein G of the rabies virus. In another advantageous embodiment, the rabies glycoprotein gene is isolated from an ERA strain.

In another embodiment, the rabies glycoprotein is any rabies glycoprotein with a known protein sequence, such as rabies virus glycoprotein G. such as the protein sequences in or derived from the nucleotide sequences in Marissen et al., J Virol. April 2005;79(8):4672-8; Dietzschold et al., Vaccine. December 9, 2004;23(4):518-24; Mansfield et al., J Gen Virol. November 2004;85(Pt 11):3279-83; Sato et al., J Vet Med Sci. July 2004;66(7):747-53; Takayama-Ito et al., J Neurovirol. April 2004;10(2):131-5; Li et al., Zhongguo Yi Xue Ke Xue Yuan Xue Bao. December 2003;25(6):650-4; Hemachudha et al., J Infect Dis. Oct. 1, 2003;188(7):960-6; Kankanamge et al., Microbiol Immunol. 2003;47(7):507-19; Maillard et al., Virus Res. June 2003;93(2):151-8; Irie et al. Microbiol Immunol. 2002;46(7):449-61; Langevin et al., J Biol Chem. Oct. 4, 2002;277(40):37655-62; Maillard and Gaudin, J Gen Virol. June 2002;83(Pt 6):1465-76; Holmes et al., Virology. Jan. 20, 2002;292(2):247-57; Mebatsion, J Virol. December 2001;75(23):11496-502; Zhang et al., Zhonghua Shi Yan He Lin Chuang Bing Du Xue Za Zhi. September 2000;14(3):281-4; Ray et al., Clin Exp Immunol. July 2001;125(1):94-101; Morimoto et al., Vaccine. May 14, 2001;19(25-26):3543-51; Morimoto et al., J Neurovirol. October 2000;6(5):373-81; Bourhy et al., J Gen Virol. October 1999;80 (Pt 10):2545-57; Kissi et al., J Gen Virol. August 1999;80 (Pt 8):2041-50; Nakahara et al., Microbiol Immunol. 1999;43(3):259-70; Matthews et al., J Gen Virol. Februrary 1999;80 (Pt 2):345-53; Tuffereau et al., EMBO J. Dec. 15, 1998;17(24):7250-9; Jallet et al. J Virol. January 1999;73(1):225-33; Wloch et al., Hum Gene Ther. Jul. 1, 1998;9(10):1439-47; Mellquist et al., Biochemistry. May 12, 1998;37(19):6833-7; Morimoto et al., Proc Natl Acad Sci U S A. Mar. 17, 1998;95(6):3152-6; Coll, Arch Virol. 1997;142(10):2089-97; Bracci et al., Blood. Nov. 1, 1997;90(9):3623-8; Gaudin et al., J Virol. November 1996;70(11):7371-8; Morimoto et al., Proc Natl Acad Sci U S A. May 28, 1996;93(11):5653-8; Mebatsion et al., Cell. Mar. 22, 1996;84(6):941-51; Shakin-Eshleman et al., J Biol Chem. Mar. 15, 1996;271(11):6363-6; Nadin-Davis et al., J Virol Methods. March 1996;57(1):1-14. Erratum in: J Virol Methods Apr. 26, 1996;58(1-2):209; Wojczyk et al., Protein Expr Purif. March 1996;7(2):183-93; Suzuki et al., J Gen Virol. December 1995;76 (Pt 12):3021-9; Raux et al., Virology. Jul. 10, 1995;210(2):400-8; Kasturi et al., J Biol Chem. Jun. 16, 1995;270(24):14756-61; Otvos et al., Biochim Biophys Acta. May 29, 1995;1267(1):55-64; Mebatsion et al., J Virol. March 1995;69(3):1444-51; Wojczyk B, Shakin-Eshleman S H, Doms R W, Xiang Z Q, Ertl H C, Wunner W H, Spitalnik, Biochemistry. Feb. 28, 1995;34(8):2599-609; Ravkov et al., Virology. Jan. 10, 1995;206(1):718-23; Ni et al., Microbiol Immunol. 1995;39(9):693-702; Coll, Arch Virol. 1995;140(5):827-51; Grabko et al., Dokl Akad Nauk. July 1994;337(1):117-21; Sakamoto et al., Virus Genes. January 1994;8(1):35-46; Fodor et al., Arch Virol. 1994;135(3-4):451-9; Ito et al., Microbiol Immunol. 1994;38(6):479-82; Shakin-Eshleman et al., Biochemistry. Sep. 14, 1993;32(36):9465-72; Morimoto et al., Virology. August 1993;195(2):541-9; van der Heijden et al., J Gen Virol. August 1993;74 (Pt 8):1539-45; Nishihara et al., Gene. Jul. 30, 1993;129(2):207-14; Rustici et al., Biopolymers. June 1993;33(6):961-9; McColl et al., Aust Vet J. March 1993;70(3):84-9; Bai et al., Virus Res. Februray 1993;27(2):101-12; Nishihara et al., Nippon Rinsho. February 1993;51(2):323-8; Bracci et al., FEBS Lett. Oct. 19, 1992;311(2):115-8; Tuchiya et al., Virus Res. Sep. 1, 1992;25(1-2):1-13; Shakin-Eshleman et al., J Biol Chem. May 25, 1992;267(15):10690-8; Whitt et al., Virology. December 1991;185(2):681-8; Benmansour et al., J Virol. August 1991;65(8):4198-203; Burger et al., J Gen Virol. February 1991;72 (Pt 2):359-67; Dietzschold et al., J Virol. August 1990;64(8):3804-9; Becker, Virus Genes. February 1990;3(3):277-84; Prehaud et al., Virology. December 1989;173(2):390-9 and Wang et al., Chin Med J (Engl). November 1989;102(11):885-9, the disclosures of which are incorporated by reference in their entireties, may be used in the present invention.

The terms “protein”, “peptide”, “polypeptide” and “polypeptide fragment” are used interchangeably herein to refer to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

In another embodiment, the rabies glycoprotein gene is any rabies glycoprotein gene with a known nucleotide sequence, such as rabies virus glycoprotein G, such as the nucleotide sequences in or derived from the protein sequences in Marissen et al., J Virol. April 2005;79(8):4672-8; Dietzschold et al., Vaccine. Dec. 9, 2004;23(4):518-24; Mansfield et al., J Gen Virol. November 2004;85(Pt 11):3279-83; Sato et al., J Vet Med Sci. July 2004;66(7):747-53; Takayama-Ito et al., J Neurovirol. April 2004;10(2):131-5; Li et al., Zhongguo Yi Xue Ke Xue Yuan Xue Bao. December 2003;25(6):650-4; Hemachudha et al., J Infect Dis. Oct. 1, 2003;188(7):960-6; Kankanamge et al., Microbiol Immunol. 2003;47(7):507-19; Maillard et al., Virus Res. June 2003;93(2):151-8; Irie et al., Microbiol Immunol. 2002;46(7):449-61; Langevin et al., J Biol Chem. Oct. 4, 2002;277(40):37655-62; Maillard and Gaudin, J Gen Virol. June 2002;83(Pt 6):1465-76; Holmes et al., Virology. Jan. 20, 2002;292(2):247-57; Mebatsion, J Virol. December 2001;75(23):11496-502; Zhang et al., Zhonghua Shi Yan He Lin Chuang Bing Du Xue Za Zhi. September 2000;14(3):281-4; Ray et al., Clin Exp Immunol. July 2001;125(l):94-101; Morimoto et al., Vaccine. May 14, 2001;19(25-26):3543-51; Morimoto et al., J Neurovirol. October 2000;6(5):373-81; Bourhy et al., J Gen Virol. October 1999;80 (Pt 10):2545-57; Kissi et al., J Gen Virol. August 1999;80 (Pt 8):2041-50; Nakahara et al., Microbiol Immunol. 1999;43(3):259-70; Matthews et al., J Gen Virol. Februray 1999;80 (Pt 2):345-53; Tuffereau et al., EMBO J. Dec. 15, 1998;17(24):7250-9; Jallet et al., J Virol. January 1999;73(1):225-33; Wloch et al., Hum Gene Ther. Jul. 1, 1998;9(10):1439-47; Mellquist et al., Biochemistry. May 12, 1998;37(19):6833-7; Morimoto et al., Proc Natl Acad Sci U S A. Mar. 17, 1998;95(6):3152-6; Coll, Arch Virol. 1997;142(10):2089-97; Bracci et al., Blood. Nov. 1, 1997;90(9):3623-8; Gaudin et al., J Virol. November 1996;70(11):7371-8; Morimoto et al., Proc Natl Acad Sci U S A. May 28, 1996;93(11):5653-8; Mebatsion et al., Cell. Mar. 22, 1996;84(6):941-51; Shakin-Eshleman et al., J Biol Chem. Mar. 15, 1996;271(11):6363-6; Nadin-Davis et al., J Virol Methods. March 1996;57(1):1-14. Erratum in: J Virol Methods Apr. 26, 1996;58(1-2):209; Wojczyk et al., Protein Expr Purif. March 1996;7(2):183-93; Suzuki et al., J Gen Virol. December 1995;76 (Pt 12):3021-9; Raux et al., Virology. Jul 10, 1995;210(2):400-8; Kasturi et al., J Biol Chem. Jun. 16, 1995;270(24):14756-61; Otvos et al., Biochim Biophys Acta. May 29, 1995;1267(1):55-64; Mebatsion et al., J Virol. March 1995;69(3):1444-51; Wojczyk B, Shakin-Eshleman S H, Doms R W, Xiang Z Q, Ertl H C, Wunner W H, Spitalnik, Biochemistry. Feb. 28, 1995;34(8):2599-609; Ravkov et al., Virology. Jan. 10, 1995;206(1):718-23; Ni et al., Microbiol Immunol. 1995;39(9):693-702; Coll, Arch Virol. 1995; 140(5):827-51; Grabko et al., Dokl Akad Nauk. July 1994;337(1):117-21; Sakamoto et al., Virus Genes. January 1994; 8(1):35-46; Fodor et al., Arch Virol. 1994;135(3-4):451-9; Ito et al., Microbiol Immunol. 1994;38(6):479-82; Shakin-Eshleman et al., Biochemistry. Sep. 14, 1993;32(36):9465-72; Morimoto et al., Virology. August 1993;195(2):541-9; van der Heijden et al., J Gen Virol. August 1993;74 (Pt 8): 1539-45; Nishihara et al., Gene. Jul. 30, 1993;129(2):207-14; Rustici et al., Biopolymers. June 1993;33(6):961-9; McColl et al., Aust Vet J. March 1993;70(3):84-9; Bai et al., Virus Res. February 1993;27(2):101-12; Nishihara et al., Nippon Rinsho. February 1993;51(2):323-8; Bracci et al., FEBS Lett. Oct. 19, 1992;311(2):115-8; Tuchiya et al., Virus Res. Sep. 1, 1992;25(1-2):1-13; Shakin-Eshleman et al., J Biol Chem. May 25, 1992;267(15):10690-8; Whitt et al., Virology. December 1991;185(2):681-8; Benmansour et al., J Virol. August 1991;65(8):4198-203; Burger et al., J Gen Virol. February 1991;72 (Pt 2):359-67; Dietzschold et al., J Virol. August 1990;64(8):3804-9; Becker, Virus Genes. February 1990;3(3):277-84; Prehaud et al., Virology. December 1989;173(2):390-9 and Wang et al., Chin Med J (Engl). November 1989;102(11):885-9, the disclosures of which are incorporated by reference in their entireties, may be used in the present invention.

A “polynucleotide” is a polymeric form of nucleotides of any length, which contain deoxyribonucleotides, ribonucleotides, and analogs in any combination. Polynucleotides may have three-dimensional structure, and may perform any function, known or unknown. The term “polynucleotide” includes double-, single-stranded, and triple-helical molecules. Unless otherwise specified or required, any embodiment of the invention described herein that is a polynucleotide encompasses both the double stranded form and each of two complementary forms known or predicted to make up the double stranded form of either the DNA, RNA or hybrid molecule.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracyl, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support.

An “isolated” polynucleotide or polypeptide is one that is substantially free of the materials with which it is associated in its native environment. By substantially free, is meant at least 50%, advantageously at least 70%, more advantageously at least 80%, and even more advantageously at least 90% free of these materials.

The invention further comprises a complementary strand to a rabies glycoprotein polynucleotide.

The complementary strand can be polymeric and of any length, and can contain deoxyribonucleotides, ribonucleotides, and analogs in any combination.

Hybridization reactions can be performed under conditions of different “stringency.” Conditions that increase stringency of a hybridization reaction are well known. See for examples, “Molecular Cloning: A Laboratory Manual”, second edition (Sambrook et al. 1989). Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%; incubation times from 5 minutes to 24 hours; 1, 2 or more washing steps; wash incubation times of 1, 2, or 15 minutes; and wash solutions of 6×SSC, 1×SSC, 0.1×SSC, or deionized water.

The invention further encompasses polynucleotides encoding functionally equivalent variants and derivatives of a rabies glycoprotein polypeptides and functionally equivalent fragments thereof which may enhance, decrease or not significantly affect properties of the polypeptides encoded thereby. These functionally equivalent variants, derivatives, and fragments display the ability to retain rabies glycoprotein activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990;87: 2264-2268, modified as in Karlin & Altschul, Proc. Natl. Acad. Sci. USA 1993;90: 5873-5877.

Another example of a mathematical algorithm used for comparison of sequences is the algorithm of Myers & Miller, CABIOS 1988;4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988;85: 2444-2448.

Advantageous for use according to the present invention is the WU-BLAST (Washington University BLAST) version 2.0 software. WU-BLAST version 2.0 executable programs for several UNIX platforms can be downloaded from ftp://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990;215: 403-410; Gish & States, 1993;Nature Genetics 3: 266-272; Karlin & Altschul, 1993;Proc. Natl. Acad. Sci. USA 90: 5873-5877; all of which are incorporated by reference herein).

In general, comparison of amino acid sequences is accomplished by aligning an amino acid sequence of a polypeptide of a known structure with the amino acid sequence of a the polypeptide of unknown structure. Amino acids in the sequences are then compared and groups of amino acids that are homologous are grouped together. This method detects conserved regions of the polypeptides and accounts for amino acid insertions and deletions. Homology between amino acid sequences can be determined by using commercially available algorithms (see also the description of homology above). In addition to those otherwise mentioned herein, mention is made too of the programs BLAST, gapped BLAST, BLASTN, BLASTP, and PSI-BLAST, provided by the National Center for Biotechnology Information. These programs are widely used in the art for this purpose and can align homologous regions of two amino acid sequences.

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983;80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. CA). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being-considered equal to uracil (U) in RNA sequences.

And, without undue experimentation, the skilled artisan can consult with many other programs or references for determining percent homology.

The invention further encompasses a rabies glycoprotein contained in a vector molecule or an expression vector and operably linked to an enhancer and/or a promoter element if necessary. In an advantageous embodiment, the promoter is a cytomegalovirus (CMV) promoter. In another embodiment, the enhancers and/or promoters include various cell or tissue specific promoters, various viral promoters and enhancers and various rabies glycoprotein DNA sequences isogenically specific for each animal species.

A “vector” refers to a recombinant DNA or RNA plasmid or virus that comprises a heterologous polynucleotide to be delivered to a target cell, either in vitro or in vivo. The heterologous polynucleotide may comprise a sequence of interest for purposes of therapy, and may optionally be in the form of an expression cassette. As used herein, a vector need not be capable of replication in the ultimate target cell or subject. The term includes cloning vectors for translation of a polynucleotide encoding sequence. Also included are viral vectors.

The term “recombinant” means a polynucleotide of genomic cDNA, semisynthetic, or synthetic origin which either does not occur in nature or is linked to another polynucleotide in an arrangement not found in nature.

“Heterologous” means derived from a genetically distinct entity from the rest of the entity to which it is being compared. For example, a polynucleotide, may be placed by genetic engineering techniques into a plasmid or vector derived from a different source, and is a heterologous polynucleotide. A promoter removed from its native coding sequence and operatively linked to a coding sequence other than the native sequence is a heterologous promoter.

The polynucleotides of the invention may comprise additional sequences, such as additional encoding sequences within the same transcription unit, controlling elements such as promoters, ribosome binding sites, polyadenylation sites, additional transcription units under control of the same or a different promoter, sequences that permit cloning, expression, homologous recombination, and transformation of a host cell, and any such construct as may be desirable to provide embodiments of this invention.

Elements for the expression of rabies glycoprotein are advantageously present in an inventive vector. In minimum manner, this comprises, consists essentially of, or consists of an initiation codon (ATG), a stop codon and a promoter, and optionally also a polyadenylation sequence for certain vectors such as plasmid and certain viral vectors, e.g., viral vectors other than poxviruses. When the polynucleotide encodes a polyprotein fragment, e.g. rabies glycoprotein, advantageously, in the vector, an ATG is placed at 5′ of the reading frame and a stop codon is placed at 3′. Other elements for controlling expression may be present, such as enhancer sequences, stabilizing sequences and signal sequences permitting the secretion of the protein.

Methods for making and/or administering a vector or recombinants or plasmid for expression of gene products of genes of the invention either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Ser. No. 920,197, filed Oct. 16,1986; WO 90/01543; WO91/11525; WO 94/16716; WO 96/39491; WO 98/33510; EP 265785; EP 0 370 573; Andreansky et al., Proc. Natl. Acad. Sci. USA 1996;93:11313-11318; Ballay et al., EMBO J. 1993;4:3861-65; Felgner et al., J. Biol. Chem. 1994;269:2550-2561; Frolov et al., Proc. Natl. Acad. Sci. USA 1996;93:11371-11377; Graham, Tibtech 1990;8:85-87; Grunhaus et al., Sem. Virol. 1992;3:237-52; Ju et al., Diabetologia 1998;41:736-739; Kitson et al., J. Virol. 1991;65:3068-3075; McClements et al., Proc. Natl. Acad. Sci. USA 1996;93:11414-11420; Moss, Proc. Natl. Acad. Sci. USA 1996;93:11341-11348; Paoletti, Proc. Natl. Acad. Sci. USA 1996;93:11349-11353; Pennock et al., Mol. Cell. Biol. 1984;4:399-406; Richardson (Ed), Methods in Molecular Biology 1995;39, “Baculovirus Expression Protocols,” Humana Press Inc.; Smith et al. (1983) Mol. Cell. Biol. 1983;3:2156-2165; Robertson et al., Proc. Natl. Acad. Sci. USA 1996;93:11334-11340; Robinson et al., Sem. Immunol. 1997;9:271; and Roizman, Proc. Natl. Acad. Sci. USA 1996;93:11307-11312. Thus, the vector in the invention can be any suitable recombinant virus or virus vector, such as a poxvirus (e.g., vaccinia virus, avipox virus, canarypox virus, fowlpox virus, raccoonpox virus, swinepox virus, etc.), adenovirus (e.g., canine adenovirus), herpesvirus, baculovirus, retrovirus, etc. (as in documents incorporated herein by reference); or the vector can be a plasmid. The herein cited and incorporated herein by reference documents, in addition to providing examples of vectors useful in the practice of the invention, can also provide sources for non- rabies glycoprotein proteins or fragments thereof, e.g., non-rabies glycoprotein proteins or fragments thereof, cytokines, etc. to be expressed by vector or vectors in, or included in, the compositions of the invention.

The present invention also relates to preparations comprising vectors, such as expression vectors, e.g., therapeutic compositions. The preparations can comprise, consist essentially of, or consist of one or more vectors, e.g., expression vectors, such as in vivo expression vectors, comprising, consisting essentially or consisting of (and advantageously expressing) one or more of a rabies glycoprotein polynucleotides and, advantageously, the vector contains and expresses a polynucleotide that includes, consists essentially of, or consists of a coding region encoding rabies glycoprotein, in a pharmaceutically or veterinarily acceptable carrier, excipient or vehicle. Thus, according to an embodiment of the invention, the other vector or vectors in the preparation comprises, consists essentially of or consists of a polynucleotide that encodes, and under appropriate circumstances the vector expresses one or more other proteins of rabies glycoprotein or a fragment thereof.

According to another embodiment, the vector or vectors in the preparation comprise, or consist essentially of, or consist of polynucleotide(s) encoding one or more proteins or fragment(s) thereof of rabies glycoprotein, the vector or vectors have express of the polynucleotide(s). The inventive preparation advantageously comprises, consists essentially of, or consists of, at least two vectors comprising, consisting essentially of, or consisting of, and advantageously also expressing, advantageously in vivo under appropriate conditions or suitable conditions or in a suitable host cell, polynucleotides from different rabies glycoprotein isolates encoding the same proteins and/or for different proteins, but advantageously for the same proteins. As to preparations containing one or more vectors containing, consisting essentially of or consisting of polynucleotides encoding, and advantageously expressing, advantageously in vivo, rabies glycoprotein, or an epitope thereof, it is advantageous that the expression products be from two, three or more different rabies glycoprotein isolates, advantageously strains. The invention is also directed at mixtures of vectors that contain, consist essentially of, or consist of coding for, and express, different rabies proteins.

In an advantageous embodiment, the vector is a viral vector, advantageously a vaccinia virus vector containing the rabies glycoprotein gene. Advantageously, the rabies glycoprotein is rabies glycoprotein G, advantageously derived from the ERA strain. In an advantageous embodiment, the vaccinia virus can be a Copenhagen strain and/or a tk⁻ phenotype. In a particularly advantageous embodiment, the vector is a vaccinia virus vector (Copenhagen strain and tk⁻ phenotype) with the rabies virus glycoprotein G encoded therein, advantageously Raboral V-RG.

In one particular embodiment the viral vector is a poxvirus, e.g. a vaccinia virus or an attenuated vaccinia virus, (for instance, MVA, a modified Ankara strain obtained after more than 570 passages of the Ankara vaccine strain on chicken embryo fibroblasts; see Stickl & Hochstein-Mintzel, Munch. Med. Wschr., 1971, 113, 1149-1153; Sutter et al., Proc. Natl. Acad. Sci. U.S.A., 1992, 89, 10847-10851; available as ATCC VR-1508; or NYVAC, see U.S. Pat. No. 5,494,807, for instance, Examples 1 to 6 and et seq of U.S. Pat. No. 5,494,807 which discuss the construction of NYVAC, as well as variations of NYVAC with additional ORFs deleted from the Copenhagen strain vaccinia virus genome, as well as the insertion of heterologous coding nucleic acid molecules into sites of this recombinant, and also, the use of matched promoters; see also WO96/40241), an avipox virus or an attenuated avipox virus (e.g., canarypox, fowlpox, dovepox, pigeonpox, quailpox, ALVAC or TROVAC; see, e.g., U.S. Pat. Nos. 5,505,941, 5,494,807), swinepox, raccoonpox, camelpox, or myxomatosis virus.

According to another embodiment of the invention, the poxvirus vector is a canarypox virus or a fowlpox virus vector, advantageously an attenuated canarypox virus or fowlpox virus. In this regard, is made to the canarypox available from the ATCC under access number VR-111. Attenuated canarypox viruses are described in U.S. Pat. No. 5,756,103 (ALVAC) and WO01/05934. Numerous fowlpox virus vaccination strains are also available, e.g. the DIFTOSEC CT strain marketed by MERIAL and the NOBILIS VARIOLE vaccine marketed by INTERVET; and, reference is also made to U.S. Pat. No. 5,766,599 which pertains to the atenuated fowlpox strain TROVAC.

For information on the method to generate recombinants thereof and how to administer recombinants thereof, the skilled artisan can refer documents cited herein and to WO90/12882, e.g., as to vaccinia virus mention is made of U.S. Pat. Nos. 4,769,330, 4,722,848, 4,603,112, 5,110,587, 5,494,807, and 5,762,938 inter alia; as to fowlpox, mention is made of U.S. Pat. Nos. 5,174,993, 5,505,941 and U.S. Pat. No. 5,766,599 inter alia; as to canarypox mention is made of U.S. Pat. No. 5,756,103 inter alia; as to swinepox mention is made of U.S. Pat. No. 5,382,425 inter alia; and, as to raccoonpox, mention is made of WO00/03030 inter alia.

When the expression vector is a vaccinia virus, insertion site or sites for the polynucleotide or polynucleotides to be expressed are advantageously at the thymidine kinase (TK) gene or insertion site, the hemagglutinin (HA) gene or insertion site, the region encoding the inclusion body of the A type (ATI); see also documents cited herein, especially those pertaining to vaccinia virus. In the case of canarypox, advantageously the insertion site or sites are ORF(s) C3, C5 and/or C6; see also documents cited herein, especially those pertaining to canarypox virus. In the case of fowlpox, advantageously the insertion site or sites are ORFs F7 and/or F8; see also documents cited herein, especially those pertaining to fowlpox virus. The insertion site or sites for MVA virus area advantageously as in various publications, including Carroll M. W. et al., Vaccine, 1997, 15 (4), 387-394; Stittelaar K. J. et al., J. Virol., 2000, 74 (9), 4236-4243; Sutter G. et al., 1994, Vaccine, 12 (11), 1032-1040; and, in this regard it is also noted that the complete MVA genome is described in Antoine G., Virology, 1998, 244, 365-396, which enables the skilled artisan to use other insertion sites or other promoters.

Advantageously, the polynucleotide to be expressed is inserted under the control of a specific poxvirus promoter, e.g., the vaccinia promoter 7.5 kDa (Cochran et al., J. Virology, 1985, 54, 30-35), the vaccinia promoter 13L (Riviere et al., J. Virology, 1992, 66, 3424-3434), the vaccinia promoter HA (Shida, Virology, 1986, 150, 451-457), the cowpox promoter ATI (Funahashi et al., J. Gen. Virol., 1988, 69, 35-47), the vaccinia promoter H6 (Taylor J. et al., Vaccine, 1988, 6, 504-508; Guo P. et al. J. Virol., 1989, 63, 4189-4198; Perkus M. et al., J. Virol., 1989, 63, 3829-3836), inter alia.

Advantageously, for the vaccination of mammals the expression vector is a canarypox or a fowlpox. In this way, there can be expression of the heterologous proteins with limited or no productive replication.

According to one embodiment of the invention, the expression vector is a viral vector, in particular an in vivo expression vector. In an advantageous embodiment, the expression vector is an adenovirus vector, such as a human adenovirus (HAV) or a canine adenovirus (CAV). Advantageously, the adenovirus is a human Ad5 vector, an E1-deleted and/or disrupted adenovirus, an E3-deleted and/or disrupted adenovirus or an E1- and E3-deleted and/or disrupted adenovirus. Optionally, E4 may be deleted and/or disrupted from any of the adenoviruses described above. For example, the human Ad5 vectors expressing a rabies glycoprotein gene described in Yarosh et al. and Lutze-Wallace et al. can be used in methods of the invention (see, e.g., Yarosh et al., Vaccine. September 1996;14(13):1257-64 and Lutze-Wallace et al., Biologicals. December 1995;23(4):271-7).

In one embodiment the viral vector is a human adenovirus, in particular a serotype 5 adenovirus, rendered incompetent for replication by a deletion in the E1 region of the viral genome. The deleted adenovirus is propagated in E1-expressing 293 cells or PER cells, in particular PER.C6 (F. Falloux et al Human Gene Therapy 1998, 9, 1909-1917). The human adenovirus can be deleted in the E3 region eventually in combination with a deletion in the E1 region (see, e.g. J. Shriver et al. Nature, 2002, 415, 331-335, F. Graham et al Methods in Molecular Biology Vol 7: Gene Transfer and Expression Protocols Edited by E. Murray, The Human Press Inc, 1991, p 109-128; Y. Ilan et al Proc. Natl. Acad. Sci. 1997, 94, 2587-2592; S. Tripathy et al Proc. Natl. Acad. Sci. 1994, 91, 11557-11561; B. Tapnell Adv. Drug Deliv. Rev.1993, 12, 185-199;X. Danthinne et al Gene Thrapy 2000, 7, 1707-1714; K. Berkner Bio Techniques 1988, 6, 616-629; K. Berkner et al Nucl. Acid Res. 1983, 11, 6003-6020; C. Chavier et al J. Virol. 1996, 70, 4805-4810). The insertion sites can be the E1 and/or E3 loci eventually after a partial or complete deletion of the E1 and/or E3 regions. Advantageously, when the expression vector is an adenovirus, the polynucleotide to be expressed is inserted under the control of a promoter functional in eukaryotic cells, such as a strong promoter, preferably a cytomegalovirus immediate-early gene promoter (CMV-IE promoter). The CMV-IE promoter is advantageously of murine or human origin. The promoter of the elongation factor 1α can also be used. In one particular embodiment a promoter regulated by hypoxia, e.g. the promoter HRE described in K. Boast et al Human Gene Therapy 1999, 13, 2197-2208), can be used. A muscle specific promoter can also be used (X. Li et al Nat. Biotechnol. 1999, 17, 241-245). Strong promoters are also discussed herein in relation to plasmid vectors. A poly(A) sequence and terminator sequence can be inserted downstream the polynucleotide to be expressed, e.g. a bovine growth hormone gene or a rabbit β-globin gene polyadenylation signal.

In another embodiment the viral vector is a canine adenovirus, in particular a CAV-2 (see, e.g. L. Fischer et al. Vaccine, 2002, 20, 3485-3497; U.S. Pat. Nos. 5,529,780; 5,688,920; PCT Application No. WO95/14102). For CAV, the insertion sites can be in the E3 region and/or in the region located between the E4 region and the right ITR region (see U.S. Pat. Nos. 6,090,393; 6,156,567). In one embodiment the insert is under the control of a promoter, such as a cytomegalovirus immediate-early gene promoter (CMV-IE promoter) or a promoter already described for a human adenovirus vector. A poly(A) sequence and terminator sequence can be inserted downstream the polynucleotide to be expressed, e.g. a bovine growth hormone gene or a rabbit β-globin gene polyadenylation signal.

In another particular embodiment the viral vector is a herpesvirus such as a canine herpesvirus (CHV) or a feline herpesvirus (FHV). For CHV, the insertion sites may be in particular in the thymidine kinase gene, in the ORF3, or in the UL43 ORF (see U.S. Pat. No. 6,159,477). In one embodiment the polynucleotide to be expressed is inserted under the control of a promoter functional in eukaryotic cells, advantageously a CMV-IE promoter (murine or human). In one particular embodiment a promoter regulated by hypoxia, e.g. the promoter HRE described in K. Boast et al Human Gene Therapy 1999, 13, 2197-2208), can be used. A poly(A) sequence and terminator sequence can be inserted downstream the polynucleotide to be expressed, e.g. bovine growth hormone or a rabbit β-globin gene polyadenylation signal.

According to a yet further embodiment of the invention, the expression vector is a plasmid vector or a DNA plasmid vector, in particular an in vivo expression vector. In a specific, non-limiting example, the pVR1020 or 1012 plasmid (VICAL Inc.; Luke C. et al., Journal of Infectious Diseases, 1997, 175, 91-97; Hartikka J. et al., Human Gene Therapy, 1996, 7, 1205-1217) can be utilized as a vector for the insertion of a polynucleotide sequence. The pVR1020 plasmid is derived from pVR1012 and contains the human tPA signal sequence.

The term plasmid covers any DNA transcription unit comprising a polynucleotide according to the invention and the elements necessary for its in vivo expression in a cell or cells of the desired host or target; and, in this regard, it is noted that a supercoiled or non-supercoiled, circular plasmid, as well as a linear form, are intended to be within the scope of the invention.

Each plasmid comprises or contains or consists essentially of, in addition to the polynucleotide encoding a rabies glycoprotein variant, analog or fragment, operably linked to a promoter or under the control of a promoter or dependent upon a promoter. In general, it is advantageous to employ a strong promoter functional in eukaryotic cells. The preferred strong promoter is the immediate early cytomegalovirus promoter (CMV-IE) of human or murine origin, or optionally having another origin such as the rat or guinea pig. The CMV-IE promoter can comprise the actual promoter part, which may or may not be associated with the enhancer part. Reference can be made to EP-A-260 148, EP-A-323 597, U.S. Pat. Nos. 5,168,062, 5,385,839, and 4,968,615, as well as to PCT Application No WO87/03905. The CMV-IE promoter is advantageously a human CMV-IE (Boshart M. et al., Cell., 1985, 41, 521-530) or murine CMV-IE.

In more general terms, the promoter has either a viral or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa M. et al., Vaccine, 2000, 18, 2337-2344), or the actin promoter (Miyazaki J. et al., Gene, 1989, 79, 269-277).

Functional sub fragments of these promoters, i.e., portions of these promoters that maintain an adequate promoting activity, are included within the present invention, e.g. truncated CMV-IE promoters according to PCT Application No. WO98/00166 or U.S. Pat. No. 6,156,567 can be used in the practice of the invention. A promoter in the practice of the invention consequently includes derivatives and sub fragments of a full-length promoter that maintain an adequate promoting activity and hence function as a promoter, preferably promoting activity substantially similar to that of the actual or full-length promoter from which the derivative or sub fragment is derived, e.g., akin to the activity of the truncated CMV-IE promoters of U.S. Pat. No. 6,156,567 to the activity of full-length CMV-IE promoters. Thus, a CMV-IE promoter in the practice of the invention can comprise or consist essentially of or consist of the promoter portion of the full-length promoter and/or the enhancer portion of the full-length promoter, as well as derivatives and sub fragments.

Advantageously, the plasmids comprise or consist essentially of other expression control elements. It is particularly advantageous to incorporate stabilizing sequence(s), e.g., intron sequence(s), preferably the first intron of the hCMV-IE (PCT Application No. WO89/01036), the intron II of the rabbit β-globin gene (van Ooyen et al., Science, 1979, 206, 337-344).

As to the polyadenylation signal (polyA) for the plasmids and viral vectors other than poxviruses, use can more be made of the poly(A) signal of the bovine growth hormone (bGH) gene (see U.S. Pat. No. 5,122,458), or the poly(A) signal of the rabbit β-globin gene or the poly(A) signal of the SV40 virus.

According to another embodiment of the invention, the expression vectors are expression vectors used for the in vitro expression of proteins in an appropriate cell system. The expressed proteins can be harvested in or from the culture supernatant after, or not after secretion (if there is no secretion a cell lysis typically occurs or is performed), optionally concentrated by concentration methods such as ultrafiltration and/or purified by purification means, such as affinity, ion exchange or gel filtration-type chromatography methods.

It is understood to one of skill in the art that conditions for culturing a host cell varies according to the particular gene and that routine experimentation is necessary at times to determine the optimal conditions for culturing rabies glycoprotein depending on the host cell. A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell and to the progeny thereof.

Polynucleotides comprising a desired sequence can be inserted into a suitable cloning or expression vector, and the vector in turn can be introduced into a suitable host cell for replication and amplification. Polynucleotides can be introduced into host cells by any means known in the art. The vectors containing the polynucleotides of interest can be introduced into the host cell by any of a number of appropriate means, including direct uptake, endocytosis, transfection, f-mating, electroporation, transfection employing calcium chloride, rubidium chloride, calcium phosphate, DEAE-dextran, or other substances; microprojectile bombardment; lipofection; and infection (where the vector is infectious, for instance, a retroviral vector). The choice of introducing vectors or polynucleotides will often depend on features of the host cell.

In an advantageous embodiment, the invention provides for the administration of a therapeutically effective amount of a formulation for the delivery and expression of rabies glycoprotein in a target cell. Determination of the therapeutically effective amount is routine experimentation for one of ordinary skill in the art. In one embodiment, the formulation comprises an expression vector comprising a polynucleotide that expresses rabies glycoprotein and a pharmaceutically or veterinarily acceptable carrier, vehicle or excipient. In an advantageous embodiment, the pharmaceutically or veterinarily acceptable carrier, vehicle or excipient facilitates transfection and/or improves preservation of the vector or protein.

The pharmaceutically or veterinarily acceptable carriers or vehicles or excipients are well known to the one skilled in the art. For example, a pharmaceutically or veterinarily acceptable carrier or vehicle or excipient can be a 0.9% NaCl (e.g., saline) solution or a phosphate buffer. Other pharmaceutically or veterinarily acceptable carrier or vehicle or excipients that can be used for methods of this invention include, but are not limited to, poly-(L-glutamate) or polyvinylpyrrolidone. The pharmaceutically or veterinarily acceptable carrier or vehicle or excipients may be any compound or combination of compounds facilitating the administration of the vector (or protein expressed from an inventive vector in vitro); advantageously, the carrier, vehicle or excipient may facilitate transfection and/or improve preservation of the vector (or protein). Doses and dose volumes are herein discussed in the general description and can also be determined by the skilled artisan from this disclosure read in conjunction with the knowledge in the art, without any undue experimentation.

The cationic lipids containing a quaternary ammonium salt which are advantageously but not exclusively suitable for plasmids, are advantageously those having the following formula: 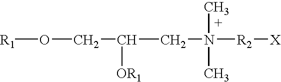
in which R₁ is a saturated or unsaturated straight-chain aliphatic radical having 12 to 18 carbon atoms, R₂ is another aliphatic radical containing 2 or 3 carbon atoms and X is an amine or hydroxyl group, e.g. the DMRIE. In another embodiment the cationic lipid can be associated with a neutral lipid, e.g. the DOPE.

Among these cationic lipids, preference is given to DMRIE (N-(2-hydroxyethyl)-N,N-dimethyl-2,3-bis(tetradecyloxy)-1-propane ammonium; WO96/34109), advantageously associated with a neutral lipid, advantageously DOPE (dioleoyl-phosphatidyl-ethanol amine; Behr J. P., 1994, Bioconjugate Chemistry, 5, 382-389), to form DMRIE-DOPE.

Advantageously, the plasmid mixture with the adjuvant is formed extemporaneously and advantageously contemporaneously with administration of the preparation or shortly before administration of the preparation; for instance, shortly before or prior to administration, the plasmid-adjuvant mixture is formed, advantageously so as to give enough time prior to administration for the mixture to form a complex, e.g. between about 10 and about 60 minutes prior to administration, such as approximately 30 minutes prior to administration.

When DOPE is present, the DMRIE:DOPE molar ratio is advantageously about 95: about 5 to about 5:about 95, more advantageously about 1: about 1, e.g., 1:1.

The DMRIE or DMRIE-DOPE adjuvant:plasmid weight ratio can be between about 50: about 1 and about 1: about 10, such as about 10: about 1 and about l:about 5, and advantageously about 1: about 1 and about 1: about 2, e.g., 1:1 and 1:2.

In a specific embodiment, the pharmaceutical composition is directly administered in vivo, and the encoded product is expressed by the vector in the host. The methods of in vivo delivery a vector encoding rabies glycoprotein (see, e.g., U.S. Pat. No. 6,423,693; patent publications EP 1052286, EP 1205551, U.S. patent publication 20040057941, WO 9905300 and Draghia-Akli et al., Mol Ther. December 2002;6(6):830-6; the disclosures of which are incorporated by reference in their entireties) can be modified to deliver a rabies glycoprotein of the present invention to a dog. The in vivo delivery of a vector encoding rabies glycoprotein described herein can be accomplished by one of ordinary skill in the art given the teachings of the above-mentioned references.

Advantageously, the pharmaceutical and/or therapeutic compositions and/or formulations according to the invention comprise or consist essentially of or consist of an effective quantity to elicit a therapeutic response of one or more expression vectors and/or polypeptides as discussed herein; and, an effective quantity can be determined from this disclosure, including the documents incorporated herein, and the knowledge in the art, without undue experimentation.

One skilled in the art can determine the effective plasmid dose to be used for each immunization or vaccination protocol and species from this disclosure and the knowledge in the art.

In an advantageous embodiment, the pharmaceutical and/or therapeutic compositions and/or formulations according to the invention are administered orally. In a particularly advantageous embodiment, the oral compositions are administered as a bait drop. For example, the bait drop can comprise a fishmeal polymer cube (1.25 inches by 0.75 inches) that is hollow. A sachet, or plastic packet, containing the rabies vaccine can be inserted into the hollow area of the bait and sealed with wax. The fishmeal is attractive to skunks and/or mongooses and strong enough to withstand distribution from airplanes flying at low altitude (e.g., about 500 feet). When a skunk or mongoose finds the bait and bites into it, the sachet ruptures, allowing the vaccine to enter the skunk's mouth. Skunks and mongooses then become vaccinated against rabies by this oral route.

Also in connection with such a therapeutic composition, from the disclosure herein and the knowledge in the art, the skilled artisan can determine the number of administrations, the administration route, and the doses to be used for each injection protocol, without any undue experimentation.

In another advantageous embodiment, the pharmaceutical and/or therapeutic compositions and/or formulations according to the invention are administered nasally. Methods of intranasal administration of vaccines in skunks are well known to one of skill in the art and may be extrapolated to mongooses (see, e.g., Rupprecht et al., J Wildl Dis. January 1990;26(1):99-102) and Tolson et al., Can J Vet Res. January 1988;52(1):58-62, the disclosures of which are incorporated by reference in their entireties).

The method includes at least one administration to an animal of an efficient amount of the therapeutic composition according to the invention. The animal may be male, female, pregnant female and newborn. This administration may be notably done by intramuscular (IM), intradermal (ID) or subcutaneous (SC) injection or via intranasal or oral administration. In an advantageous embodiment, the administration is oral, advantageously as a bait drop formulation. In an alternate embodiment, the therapeutic composition according to the invention can be administered by a syringe or a needleless apparatus (like for example Pigjet, Biojector or Vitajet (Bioject, Oreg., USA)). Another approach to administer plasmid is to use electroporation see, e.g. S. Tollefsen et al. Vaccine, 2002, 20, 3370-3378; S. Tollefsen et al. Scand. J. Immunol., 2003, 57, 229-238; S. Babiuk et al., Vaccine, 2002, 20, 3399-3408; PCT Application No. WO99/01158.

The invention relates to the use of the pharmaceutical compositions for the treatment of rabies in wild animals, advantageously skunks and/or mongooses. The safety of an oral vaccine for rabies, e.g., Raboral V-RG, has already been tested in striped skunks necessary to satisfy USDA regulations (see, e.g., Mackowiak et al., Adv Vet Med. 1999;41:571-83, the disclosure of which is incorporated by reference in its entirety).

While the invention has been described with reference to specific methods and embodiments, such description is for illustrative purposes only. The words used are words of description rather than of limitation. It is to be understood that changes and variations may be made by those of ordinary skill in the art without departing from the spirit or scope of the present invention, which is set forth in the following claims.

The invention will now be further described by way of the following non-limiting examples.

### EXAMPLES

### Example 1

### Skunk Challenge Results (116 days) 16.6 Weeks Post Challenge

Skunks were immunized with a dosage of 10^{8.0} TCID₅₀/1.5 ml of Raboral V-RG by the oral route or in a coated sachet using a dosage comparable to that used successfully to immunize raccoons. The skunks were challenged with rabies virus by injecting 0.5 ml into each masseter muscle. Rabies challenge virus R98-0100 AB (log 10^{6.3} MICLD₅₀/ml) was diluted 1:25 with 2% horse serum in PBS after immunization.

Results of the challenge study are as follows: Seven (7) of seven (7) non-immunized controls were dead three weeks after challenge indicating a mortality rate of 100%. Four (4) of five (5) skunks eating one (1) coated sachet containing VRG virus (10^{8.0} per dose) died by week 6 post-challenge, as did 100% of the Five (5) of five (5) skunks eating three (3) coated sachet containing VRG (10^{8.0} per dose). However, four (4) of six (6) skunks receiving 10^{8.0} Raboral V-RG per dose given by direct instillation via the oral route survived this stringent challenge (33% mortality). These results indicate that Raboral V-RG elicits a protective immune response against rabies in skunks when administered via an oral route.

### Example 2

### Efficacy of A Vaccinia Vectored Oral Rabies Vaccine in Striped Skunks

(Mephitis mephitis)

Rabies is a fatal viral encephalitic infection that affects both wild and domestic mammals and is transmissible to humans. Striped skunk (Mephitis mephitis) and raccoon (Procyon rotor) populations are major wildlife rabies reservoirs in the eastern United States (U.S.), possibly sharing epizootic cycles via spillover of species-specific variants (Guerra et al., 2003, Emerging Infectous Diseases 9:1143-1150). In California and the central United States, three rabies variants are responsible for this disease in skunks (Krebs, et al., 2004, Journal of the American Veterinary Medical Association 225:1837-1849). In Europe, an orally administered recombinant poxvirus, V-RG, has been shown to be an effective vaccine in controlling red fox (Vulpes vulpes) rabies (Brochier et al., 2001, Ann Med Vet 145:293-305). This same vaccinia-based oral vaccine, contained inside fishmeal polymer baits, shows promise in controlling rabies in U.S. raccoon populations (Hanlon et al., 1998, Journal of Wildlife Diseases 34:228-239). It has also contributed to the elimination of coyote rabies in southern Texas (Fearneyhough, et al., 1998, Journal of the American Veterinary Medical Association 212:498-502). Control of rabies in skunk populations, however, continues to be an elusive goal.

Modified-live rabies vaccines, historically used in Europe and Canada to control fox rabies, are ineffective and potentially pathogenic in skunks (Rupprecht et al., 1990, Journal of Wildlife Diseases 26:99-102; Tolson et al., 1990, Canadian Journal of Veterinary Research 54: 178-183). The vaccinia-vectored rabies vaccine is safe in this species but has been suggested to be ineffective (Charlton et al., 1992, Archives of Virology 123:169-179). This statement is in contrast to a study in which efficacy was demonstrated in skunks given relatively high titers (10^{9.0} pfu/dose) of virus by multiple routes (Tolson et al., 1987, Canadian Journal of Veterinary Research 51:363-366). The present study was conducted to determine if a commercial serial of the recombinant vaccinia virus, when given by the oral route, could protect caged skunks against a virulent rabies challenge. The raccoon field dose of 10^{7.7} TCID₅₀/ml was chosen, contained in a 1.5 to 2.0 ml volume, given by direct instillation and within a coated sachet. Caged skunks were used to allow direct observation of bait consumption. The efficacy of direct oral instillation of the vaccine was compared to the efficacy of the vaccine delivered within a bait.

Twenty-three (23) adult striped skunks (Mephitis mephitis) between the ages of 1 and 5 years, obtained from a commercial source (Ruby's Fur Farm, New Sharon, Iowa, USA) were housed individually in stainless steel cages, offered a commercial feline ration and provided with water ad libitum. After an acclimation period of approximately 2 months, during which they were accustomed to various bait formats, the animals were randomly assigned to one of four treatment groups. One group of seven skunks remained unvaccinated. Vaccinated skunks received 1.5 to 2.0 ml of a production serial of (Rabies Vaccine, Live Vaccinia Vector; trade name: Raboral V-RG®, Merial Limited, Athens, Ga.) containing 10^{7.7} TCID₅₀/ml. Two groups of five skunks each were offered either a single coated sachet or a total of three sachets, given as individual doses, on three consecutive days. Another group of six skunks received 1.5 ml dose of vaccine, equivalent to the contents of a single sachet, by oral instillation via a 3.0 ml needle-free syringe while under light sedation by the intramuscular administration of medetomidine hydrochloride (0.02 mg/kg) ketamine hydrochloride (5 mg/kg). Swallowing reflexes were observed in skunks receiving the vaccine by direct instillation. Skunks consuming sachets were observed and scored for bait acceptance. Vaccine titer was confirmed post-administration by titration in cell culture. Blood samples were collected via the jugular vein of sedated skunks 3 days prior to vaccination, 32 days post-vaccination and on the day of challenge (116 days post-vaccination). Sera were evaluated for rabies virus neutralizing antibodies (VNA) using the Rapid Fluorescent Focus Inhibition Test (RFFIT) (Smith et al., 1996, A rapid fluorescent focus inhibition test (RFFIT) for determining rabies virus-neutralizing antibody. In Laboratory techniques in rabies, 4^{th} Edition, F.-X. Meslin, M. M. Kaplan and H. Koprowski (eds.). World Health Organization, Geneva, Switzerland, pp. 181-92). For rabies challenge, each skunk was administered the rabies virus challenge material by intramuscular injection of 0.5 ml rabies virus stock (Skunk isolate, strain R98-0100, log 10^{6.3} MICLD₅₀/ml) bilaterally, into each masseter muscle. Skunks were observed daily for 56 days post-challenge for clinical signs of rabies. Animals were euthanized by the intracardiac injection of sodium pentobarbital (300 mg/kg) following intramuscular administration of ketamine hydrochloride (2.2 mg/kg) and acepromazine maleate (0.02 mg/kg). The diagnosis of rabies was confirmed post-mortem by subjecting brain tissue to direct immunofluorescent staining with anti-rabies virus monoclonal antibody (Velleca and Forrester, 1981, Detection and identification. In Laboratory methods for detection rabies. U.S. Department of Health and Human Services, Public Health Service, Centers for disease control, Atlanta, Ga., pp. 69-107). Collection of blood samples and administration of the challenge virus was performed under heavy sedation following intramuscular administration of 0.04-mg/kg medetomidine hydrochloride (Pfizer Animal Health, Inc., Westchester, Pa., USA) and 10 mg/kg ketamine hydrochloride (Fort Dodge Laboratories, Inc., Fort Dodge, Iowa, USA). All animal care and experimental procedures were performed in compliance with established Institutional Animal Care and Use Guidelines.

The RFFIT data and protection against rabies challenge results are summarized in Table 1. Six of six (100%) naive skunks succumbed to challenge (mean survival time=17 days post-challenge). Four of six (67%) skunks that received the vaccine by oral instillation survived challenge. In this group, rabies VNA were present in the four surviving skunks at 32 days post-vaccination (GMT=1.2), and declined to baseline or residual levels (GMT=0.40) by the day of challenge (116 days post-vaccination). The two skunks in this group that did not survive challenge (mean survival time=16 days), failed to seroconvert following vaccination. One of those skunks (S19) was noted to have received less than the full dose of vaccine due to insufficient sedation. All ten skunks that were offered the coated sachets readily accepted the bait. Acceptance was scored as consumption of the entire sachet (i.e. no part remaining in the cage), or puncturing of the plastic material and absence of vaccine contents. Rabies VNAs were not detected in the sera of five skunks ingesting multiple doses of the vaccine offered in a coated sachet nor did any of this group survive challenge (0 of 5, or 0% survival with a mean survival time of 21 days, range=17 to 26 days). Likewise, none of the five skunks ingesting a single sachet developed VNA against rabies. However, in this group one skunk (1 of 5, or 20%) survived rabies challenge; suggesting that sufficient vaccine was consumed to elicit immunity. Brain tissues from 18 rabies-suspect skunks were positive for reactivity with rabies virus monoclonal antibody by direct fluorescence. Brain tissue samples collected from the five surviving skunks at 56 days post-challenge were negative for detection of rabies virus antigens.

This study showed that direct oral instillation of Raboral V-RG® at 10^{7.7} TCID₅₀/1.5 ml dose protected 67% skunks against a virulent rabies virus challenge. The vaccine was immunogenic and efficacious in a small group of domestically raised skunks at a titer used for field application in raccoons. Although ten skunks readily consumed the fishmeal-coated sachets, subsequent challenge of these animals revealed poor vaccine delivery efficiency whether one or three sachets were eaten (i.e., 90% and 100% mortality, respectively). In this study, as well as during the red fox vaccine field trials in Europe (Brochier et al. 1990, Veterinary Record 127:165-167), it was shown that vaccine delivery directly impacts the evaluation of oral vaccine efficacy. These results demonstrate the value of using direct instillation to evaluate oral vaccines in a target species. Evaluation of an oral vaccine in a chosen bait is critical for field efficacy but post-baiting serology and rabies prevalence data remain indirect measures of vaccine efficacy.

The vaccinia vectored oral rabies vaccine, RABORAL V-RG® as formulated for use in raccoons, is capable of protecting a percentage of skunks against rabies. V-RG may prove to be an effective oral rabies vaccine for striped skunks. The logistical advantage of distributing one vaccine into the environment to immunize both raccoons and skunks is an obvious cost-savings to this approach. Field studies in wild populations of skunks using vaccine-filled coated sachets will provide additional data as to the suitability of this bait format for this species.

TABLE 1
Rabies virus-neutralizing antibodies and protection from
rabies challenge in skunks following uptake of V-RG vaccine
by direct oral instillation and bait acceptance.

| | | | | | Response |
|---|---|---|---|---|---|
| | Skunk | Rabies Virus Antibody Titer * | | | to Rabies |
| Group | ID | Day −3 | Day 32 | Day 116 | Challenge |
| Unvaccinated | S11 | 0.2 | 0.2 | 0.3 | R (15) |
| | S12 | 0.2 | 0.2 | 2.2 | R (15) |
| | S14 | 0.2 | 0.2 | 0.3 | R (20) |
| | S15 | 0.2 | 0.2 | 0.3 | R (15) |
| | S16 | 0.2 | 0.2 | 0.3 | R (20) |
| | S2 | 0.2 | 0.2 | 0.3 | R (18) |
| | S24 | 0.2 | 0.2 | 0.3 | R (18) |
| Oral | S19 | 0.2 | 0.2 | 0.3 | R (15) |
| Instillation | S23 | 0.2 | 0.5 | 0.3 | S |
| | S3 | 0.2 | 2.1 | 0.6 | S |
| | S5 | 0.2 | 3.9 | 0.3 | S |
| | S6 | 0.2 | 0.4 | 0.5 | S |
| | S9 | 0.2 | 0.2 | 0.3 | R (18) |
| Single Sachet | S1 | 0.2 | 0.2 | 0.3 | R (21) |
| | S13 | 0.2 | 0.2 | 0.3 | R (16) |
| | S18 | 0.2 | 0.2 | 0.3 | R (19) |
| | S4 | 0.2 | 0.2 | 0.3 | R (22) |
| | S8 | 0.2 | 0.2 | 0.3 | S |
| Multiple | S10 | 0.2 | 0.2 | 0.3 | R (25) |
| Sachets (3) | S17 | 0.2 | 0.2 | 0.3 | R (26) |
| | S20 | 0.2 | 0.2 | 0.3 | R (15) |
| | S21 | 0.2 | 0.2 | 0.3 | R (17) |
| | S22 | 0.2 | 0.2 | 0.3 | R (20) |

* Results are expressed in IU/ml.
S = survived, R = died or euthanized following signs of rabies (day of death/euthanasia following challenge).

The invention is further described by the following numbered paragraphs:

1. A method of eliciting an immune response in a skunk or mongoose comprising administering a composition comprising a viral vector comprising a rabies surface glycoprotein gene inserted into the viral vector genome in an amount effective for eliciting an immune response in the skunk or mongoose.

2. The method of paragraph 1 wherein the vector comprises a modified live vaccinia virus.

3. The method of paragraph 1 wherein the rabies surface glycoprotein gene is rabies glycoprotein G.

4. The method of paragraph 3 wherein the rabies glycoprotein G is derived from an ERA strain.

5. The method of paragraph 2 wherein the vaccinia virus is a Copenhagen strain.

6. The method of paragraph 2 wherein the vaccinia virus has a tk- phenotype.

7. The method of paragraph 2 wherein the vaccinia virus is a Copenhagen strain and has a tk⁻ phenotype.

8. The method of paragraph 2 wherein the modified live vaccinia virus is Raboral V-RG.

9. The method of paragraphs 1 to 8 wherein the administration is oral.

10. The method of paragraph 9 wherein the oral administration is by a bait drop.

11. The method of paragraph 10 wherein the bait drop comprises a hollow polymer cube.

12. The method of paragraph 11 wherein the composition is inserted in the hollow polymer cube.

13. A method for inducing an immunological or protective response in a skunk or a mongoose comprising administering a composition comprising a viral vector comprising a rabies surface glycoprotein gene inserted into the viral vector genome in an amount effective for inducing the response in the skunk or mongoose.

14. The method of paragraph 13 wherein the vector comprises a modified live vaccinia virus.

15. The method of paragraph 13 wherein the rabies surface glycoprotein gene is rabies glycoprotein G.

16. The method of paragraph 15 wherein the rabies glycoprotein G is derived from an ERA strain.

17. The method of paragraph 14 wherein the vaccinia virus is a Copenhagen strain.

18. The method of paragraph 14 wherein the vaccinia virus has a tk⁻ phenotype.

19. The method of paragraph 14 wherein the vaccinia virus is a Copenhagen strain and has a tk⁻ phenotype.

20. The method of paragraph 14 wherein the modified live vaccinia virus is Raboral V-RG.

21. The method of paragraphs 13 to 20 wherein the administration is oral.

22. The method of paragraph 21 wherein the oral administration is by a bait drop.

23. The method of paragraph 22 wherein the bait drop comprises a hollow polymer cube.

24. The method of paragraph 23 wherein the composition is inserted in the hollow polymer cube.

25. The method of any one of paragraphs 1 to 24 wherein the immune, immunological or protective response is in a skunk.

26. The method of any one of paragraphs 1 to 24 wherein the immune, immunological or protective response is in a mongoose.

27. A kit for performing the method of any one of paragraphs 1 to 26 comprising the composition of paragraphs 1 to 26 and instructions for performing the method.

Having thus described in detail advantageous embodiments of the present invention, it is to be understood that the invention defined by the above paragraphs is not to be limited to particular details set forth in the above description as many apparent variations thereof are possible without departing from the spirit or scope of the present invention.

1. A method of eliciting an immune response in a skunk or mongoose comprising administering a composition comprising a viral vector comprising a rabies surface glycoprotein gene inserted into the viral vector genome in an amount effective for eliciting an immune response in the skunk or mongoose.

2. The method of claim 1 wherein the vector comprises a modified live vaccinia virus.

3. The method of claim 1 wherein the rabies surface glycoprotein gene is rabies glycoprotein G.

4. The method of claim 3 wherein the rabies glycoprotein G is derived from an ERA strain.

5. The method of claim 2 wherein the vaccinia virus is a Copenhagen strain.

6. The method of claim Z wherein the vaccinia virus has a tk⁻ phenotype.

7. The method of claim 2 wherein the vaccinia virus is a Copenhagen strain and has a tk⁻ phenotype.

8. The method of claim 2 wherein the modified live vaccinia virus is Raboral V-RG.

9. The method of claim 1 wherein the administration is oral.

10. The method of claim 9 wherein the oral administration is by a bait drop.

11. The method of claim 10 wherein the bait drop comprises a hollow polymer cube.

12. The method of claim 11 wherein the composition is inserted in the hollow polymer cube.

13. The method of claim 1 wherein the immune response is elicited in a skunk.

14. The method of claim 1 wherein the immune response is elicited in a mongoose.
